(19) United States
(12) Patent Application Publication
(10) Pub. No.: US 2022/0251580 A1
(43) Pub. Date: Aug. 11, 2022

(54) IMPROVED GENE EDITING SYSTEM

(71) Applicant: Institute of Genetics and Developmental Biology, Chinese Academy of Sciences, Beijing (CN)

(72) Inventors: Caixia Gao, Beijing (CN); Huawei Zhang, Beijing (CN); Shengxing Wang, Beijing (CN)

(73) Assignee: Institute of Genetics and Developmental Biology, Chinese Academy of Sciences, Beijing (CN)

(21) Appl. No.: 17/609,640

(22) PCT Filed: May 7, 2020
(86) PCT No.: PCT/CN2020/088887

§ 371 Date: Nov. 8, 2021

(30) Foreign Application Priority Data
May 7, 2019 (CN) 201910375061.9

(51) Int. Cl. C12N 15/79; C12N 15/90; C12N 9/24; C12N 9/88

(52) U.S. Cl. CPC C12N 15/79; C12N 15/90; C12N 2310/20; C12N 9/88; C12Y 305/04001; C12N 9/2497

(57) ABSTRACT

Provided is a gene editing system for editing a target sequence in the genome of a cell, comprising a CRISPR nuclease, a cytosine deaminase, an AP lyase, a guide RNA and optionally an uracil-DNA glycosylase. Also provided are a method of producing a genetically modified cell, and a kit comprising the gene editing system.

### TECHNICAL FIELD

The invention relates to the field of genetic engineering. In particular, the present invention relates to an improved gene editing system. More specifically, the present invention relates to a gene editing system capable of providing accurate editing, particularly predictable accurate polynucleotide deletion, to the genome of a eukaryotic cell.

### BACKGROUND

In recent years, as the continuous development of genome editing technology, a large number of gene editing tools have been developed, improved and applied, from the gene knockout tools mediated by SpCas9 to the single-base editing tools mediated by nCas9 (D10A) fusion cytosine deaminase, etc. Under the guidance of the guide RNA, SpCas9 binds and cleaves double-stranded DNA to form a double-stranded break (DSB). In the repair process of organism, insertions and/or deletions of fragment with different lengths are often introduced. However, such insertions and/or deletions are random, inaccurate and unpredictable (Wang et al., 2014; Zhang et al., 2016). Cermak et al. (2017) significantly increased the frequency of deletion mutations and the length of the deletion fragments by Cas9 fusion 3′ repair exonuclease 2 (Trex2), but the mutation type is still inaccurate and unpredictable. Targeted deletion using a pair of sgRNAs may result in deletion of a specific long fragment, but at the same time it also produces inversion, small fragments of InDel, etc., which also greatly reduces the efficiency of the former (Cermak et al., 2017). In order to provide precise fragment deletions, Wolfs et al. (2016) fused Cas9 with TevI nuclease, which recognizes the enzymatic cleavage site and cleaves the double-stranded DNA. This cleavage forms 33-36 bp deletion together with the DSB cleaved by Cas9. However, due to the restriction of the cleavage site, the efficiency of this system is low. Up till now, a tool capable of providing efficient, accurate, and predictable short fragment deletion within the protospacer has still not been developed.

As such, it is still desirable in the art for a gene editing system capable of providing accurate editing, particularly predictable accurate polynucleotide deletion, to the genome of a eukaryotic cell.

### SUMMARY OF THE INVENTION

In one aspect, the present invention provides a gene editing system for editing a target sequence in the genome of a cell, comprising:

i) a first polypeptide and/or an expression construct comprising a nucleotide sequence encoding the first polypeptide;

ii) a second polypeptide and/or an expression construct comprising a nucleotide sequence encoding the second polypeptide; and

iii) a guide RNA and/or an expression construct comprising a nucleotide sequence encoding the guide RNA,

wherein the first polypeptide comprises CRISPR nuclease, cytosine deaminase and optionally uracil-DNA glycosylase (UDG), the second polypeptide comprises AP lyase, wherein the guide RNA is capable of targeting the first polypeptide to the target sequence in the genome of the cell.

In one aspect, the present invention provides a gene editing system for editing a target sequence in a cell genome, comprising:

i) a polypeptide and/or an expression construct comprising a nucleotide sequence encoding the polypeptide; and

ii) a guide RNA and/or an expression construct comprising a nucleotide sequence encoding the guide RNA,

wherein the polypeptide comprises CRISPR nuclease, cytosine deaminase, AP lyase and optionally uracil-DNA glycosylase (UDG), wherein the guide RNA is capable of targeting the polypeptide to the target sequence in the cell genome.

In an aspect, the present invention provides a method for producing a genetically modified cell, comprising introducing the gene editing system of the present invention into the cell.

In an aspect, the present invention provides a kit comprising the gene editing system of the invention and instructions for use.

### BRIEF DESCRIPTION OF THE DRAWINGS

FIG. 1 shows the working mode of an ACD system.

FIG. 2 shows a comparative analysis of the efficiency of InDel generation at different targeting sites between SpCas9 and ACD systems.

FIG. 3 shows the type and efficiency of the deletion mutation formed by the ACD system at the sgF3HT4 site.

FIG. 4 shows the type and efficiency of the deletion mutation formed by the ACD system at the sgLART4 site.

FIG. 5 shows the type and efficiency of the deletion mutation formed by the ACD system at the sgMYBT2 site.

FIG. 6 shows the type and efficiency of the deletion mutation formed by the ACD system at the sgPMKT1 site.

FIG. 7 shows the type and efficiency of the deletion mutation formed by the ACD system at the sgVRN1T1 site.

FIG. 8 shows the type and efficiency of the deletion mutation formed by the ACD system at the sgGS6T2 site.

FIG. 9. shows the difference in deamination activity and deamination window of different cytosine deaminase.

FIG. 10. shows the schematic diagram of the vector construction of two different types of AFID systems.

FIG. 11. shows the deletion efficiency of Cas9, AFID-3, and eAFID-3 on different rice endogenous targets.

FIG. 12. shows the deletion efficiency of Cas9, AFID-3, and eAFID-3 on different wheat endogenous targets.

FIG. 13. shows types and proportions of deletion mutations of AFID-3 and eAFID-3 on rice endogenous targets.

FIG. 14. shows types and proportions of deletion mutations of AFID-3 and eAFID-3 on wheat endogenous targets.

FIG. 15. shows the preference of AFID-3 and eAFID-3 for cytosine bases where the predictable fragment deletion starts.

FIG. 16. shows that the mutation types and proportions thereof of required predictable deletion in the reading frame generated by the Cas9, AFID-3, and eAFID-3 at the miR396h binding site of the rice OsGRF1 gene and the miR156 binding site of the OsIPA1 gene, respectively.

FIG. 17. shows the schematic diagram of the construction of the AFID-3 vector used for Agrobacterium infection in rice.

FIG. 18. shows the types of regenerated plant mutants produced by Cas9 and AFID-3 on the rice OsCDC48 gene.

### DETAILED DESCRIPTION OF THE INVENTION

### I. Definition

In the present invention, the scientific and technical terms used herein have the meaning as commonly understood by a person skilled in the art unless otherwise specified. Also, the protein and nucleic acid chemistry, molecular biology, cell and tissue culture, microbiology, immunology related terms, and laboratory procedures used herein are terms and routine steps that are widely used in the corresponding field. For example, standard recombinant DNA and molecular cloning techniques used in the present invention are well known to those skilled in the art and are more fully described in the following document: Sambrook, J., Fritsch, E. F. and Maniatis, T., Molecular Cloning: A Laboratory Manual; Cold Spring Harbor Laboratory Press: Cold Spring Harbor, 1989 (hereinafter referred to as “Sambrook”).

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

When the term “comprise” is used herein to describe the sequence of a protein or nucleic acid, the protein or nucleic acid may consist of the sequence, or may have additional amino acids or nucleotide at one or both ends of the protein or nucleic acid, but still have the activity described in this invention. In addition, those skilled in the art know that the methionine encoded by the start codon at the N-terminus of the polypeptide will be retained under certain practical conditions (for example, when expressed in a specific expression system), but does not substantially affect the function of the polypeptide. Therefore, when describing the amino acid sequence of specific polypeptide in the specification and claims of the present application, although it may not include the methionine encoded by the start codon at the N-terminus, the sequence containing the methionine is also encompassed, correspondingly, its coding nucleotide sequence may also contain a start codon; vice versa.

“Genome” as used herein encompasses not only chromosomal DNA present in the nucleus, but also organellar DNA present in the subcellular components (eg, mitochondria, plastids) of the cell.

As used herein, “organism” includes any organism, preferably eukaryotic organism that is suitable for genomic editing. Examples of organisms include, but are not limited to, mammals such as humans, mice, rats, monkeys, dogs, pigs, sheep, cattle, cats; poultry such as chickens, ducks, geese; plants including monocots and dicots such as rice, corn, wheat, sorghum, barley, soybean, peanut, arabidopsis and the like.

A “genetically modified organism” or “genetically modified cell” includes the organism or the cell which comprises within its genome an exogenous polynucleotide or a modified gene or expression regulatory sequence. For example, the exogenous polynucleotide is stably integrated within the genome of the organism or the cell such that the polynucleotide is passed on to successive generations. The exogenous polynucleotide may be integrated into the genome alone or as part of a recombinant DNA construct. The modified gene or expression regulatory sequence means that, in the organism genome or the cell genome, said sequence comprises one or more nucleotide substitution, deletion, or addition.

“Exogenous” in reference to a sequence means a sequence from a foreign species, or refers to a sequence in which significant changes in composition and/or locus occur from its native form through deliberate human intervention if from the same species.

“Polynucleotide”, “nucleic acid sequence”, “nucleotide sequence” or “nucleic acid fragment” are used interchangeably and are single-stranded or double-stranded RNA or DNA polymers, optionally containing synthetic, non-natural or altered nucleotide bases. Nucleotides are referred to by their single letter names as follows: “A” is adenosine or deoxyadenosine (corresponding to RNA or DNA, respectively), “C” means cytidine or deoxycytidine, “G” means guanosine or deoxyguanosine, “U” represents uridine, “T” means deoxythymidine, “R” means purine (A or G), “Y” means pyrimidine (C or T), “K” means G or T, “H” means A or C or T, “I” means inosine, and “N” means any nucleotide.

“Polypeptide,” “peptide,” and “protein” are used interchangeably in the present invention to refer to a polymer of amino acid residues. The terms apply to an amino acid polymer in which one or more amino acid residues is artificial chemical analogue of corresponding naturally occurring amino acid(s), as well as to a naturally occurring amino acid polymer. The terms “polypeptide,” “peptide,” “amino acid sequence,” and “protein” may also include modified forms including, but not limited to, glycosylation, lipid ligation, sulfation, y carboxylation of glutamic acid residues, and ADP-ribosylation.

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Although there are many methods for measuring the identity between two polynucleotides or polypeptides, the term “identity” is well known to the skilled person (Carrillo, H. & Lipman, D., SIAM J Applied Math 48: 1073 (1988)).

Suitable conserved amino acid replacements in peptides or proteins are known to those skilled in the art and can generally be carried out without altering the biological activity of the resulting molecule. In general, one skilled in the art recognizes that a single amino acid replacement in a non-essential region of a polypeptide does not substantially alter biological activity (See, for example, Watson et al., Molecular Biology of the Gene, 4th Edition, 1987, The Benjamin/Cummings Pub. co., p. 224).

As used in the present invention, “expression construct” refers to a vector such as a recombinant vector that is suitable for expression of a nucleotide sequence of interest in an organism. “Expression” refers to the production of a functional product. For example, expression of a nucleotide sequence may refer to the transcription of a nucleotide sequence (eg, transcription to produce mRNA or functional RNA) and/or the translation of an RNA into a precursor or mature protein.

The “expression construct” of the present invention may be a linear nucleic acid fragment, a circular plasmid, a viral vector or, in some embodiments, an RNA that is capable of translation (such as mRNA).

The “expression construct” of the present invention may comprise regulatory sequences and nucleotide sequences of interest from different origins, or regulatory sequences and nucleotide sequences of interest from the same source but arranged in a manner different from that normally occurring in nature.

“Regulatory sequence” and “regulatory element” are used interchangeably to refer to a nucleotide sequence that is located upstream (5 ‘non-coding sequence), middle or downstream (3’ non-coding sequence) of a coding sequence and affects the transcription, RNA processing or stability or translation of the relevant coding sequence. Regulatory sequences may include, but are not limited to, promoters, translation leaders, introns and polyadenylation recognition sequences.

“Promoter” refers to a nucleic acid fragment capable of controlling the transcription of another nucleic acid fragment. In some embodiments of the present invention, the promoter is a promoter capable of controlling the transcription of a gene in a cell, whether or not it is derived from the cell. The promoter may be a constitutive promoter or tissue-specific promoter or developmentally-regulated promoter or inducible promoter.

“Constitutive promoter” refers to a promoter that may in general cause the gene to be expressed in most cases in most cell types. “Tissue-specific promoter” and “tissue-preferred promoter” are used interchangeably and mean that they are expressed primarily but not necessarily exclusively in one tissue or organ, but also in a specific cell or cell type. “Developmentally-regulated promoter” refers to a promoter whose activity is dictated by developmental events. “Inducible promoter” selectively express operably linked DNA sequences in response to an endogenous or exogenous stimulus (environment, hormones, chemical signals, etc.).

Examples of promoters include, but are not limited to, the polymerase (pol) I, pol II or pol III promoters. Examples of the pol I promoter include the gallus RNA pol I promoter. Examples of the pol II promoters include, but are not limited to, the immediate-early cytomegalovirus (CMV) promoter, the Rous sarcoma virus long terminal repeat (RSV-LTR) promoter, and the immediate-early simian virus 40 (SV40) promoter. Examples of pol III promoters include the U6 and H1 promoters. An inducible promoter such as a metallothionein promoter can be used. Other examples of promoters include the T7 phage promoter, the T3 phage promoter, the β-galactosidase promoter, and the Sp6 phage promoter, and the like. Promoters that can be used in plants include, but are not limited to, cauliflower mosaic virus 35S promoter, maize Ubi-1 promoter, wheat U6 promoter, rice U3 promoter, and rice actin promoter, and the like.

As used herein, the term “operably linked” refers to the linkage of a regulatory element (e.g., but not limited to, a promoter sequence, a transcription termination sequence, etc.) to a nucleic acid sequence (e.g., a coding sequence or an open reading frame) such that transcription of the nucleotide sequence is controlled and regulated by the transcriptional regulatory element. Techniques for operably linking regulatory element regions to nucleic acid molecules are known in the art.

“Introduction” of a nucleic acid molecule (e.g., plasmid, linear nucleic acid fragment, RNA, etc.) or protein into an organism means that the nucleic acid or protein is used to transform an organism cell such that the nucleic acid or protein is capable of functioning in the cell. As used in the present invention, “transformation” includes both stable and transient transformations.

“Stable transformation” refers to the introduction of exogenous nucleotide sequences into the genome, resulting in the stable inheritance of foreign genes. Once stably transformed, the exogenous nucleic acid sequence is stably integrated into the genome of the organism and any of its successive generations.

“Transient transformation” refers to the introduction of a nucleic acid molecule or protein into a cell, performing a function without the stable inheritance of an exogenous gene. In transient transformation, the exogenous nucleic acid sequences are not integrated into the genome.

### II. Improved Gene Editing System

The present inventors have surprisingly discovered that accurate deletion from the DSB site in the target sequence to the C nucleotide site may be provided through targeting the CRISPR nuclease to the target sequence in the cell genome by the guide RNA to form a double-stranded break (DSB), while converting the C in the target sequence or its complementary sequence to U by the cytosine deaminease fused with the CRISPR nuclease, and then through the combined effect of the endogenous or exogenous uracil-DNA glycosylase (UDG) and AP lyase.

In one aspect, the present invention provides a gene editing system for editing a target sequence in the genome of a cell, comprising:

i) a first polypeptide and/or an expression construct comprising a nucleotide sequence encoding the first polypeptide;

ii) a second polypeptide and/or an expression construct comprising a nucleotide sequence encoding the second polypeptide; and

iii) a guide RNA and/or an expression construct comprising a nucleotide sequence encoding the guide RNA,

wherein the first polypeptide comprises cytosine deaminase, CRISPR nuclease and optionally uracil-DNA glycosylase (UDG), and the second polypeptide comprises AP lyase, wherein the guide RNA is capable of targeting the first polypeptide to the target sequence in the genome of the cell. In some embodiments, the expression construct comprising the nucleotide sequence encoding the first polypeptide, the expression construct comprising the nucleotide sequence encoding the second polypeptide, and/or the expression construct comprising the nucleotide sequence encoding the guide RNA may be different expression constructs, or any two or all of them may be the same expression construct. In some embodiments, the first polypeptide is isolated, and the second polypeptide is isolated polypeptide and/or the guide RNA is isolated RNA.

As used herein, “gene editing system” refers to a combination of components required for gene editing of a genome in a cell. The various components of the system, such as polypeptides, gRNA, etc., may exist independently of each other, or may exist in any combination thereof.

In some embodiments, the gene editing system comprises at least an expression construct comprising a nucleotide sequence encoding the first polypeptide, a nucleotide sequence encoding a self-cleaving peptide, and a nucleotide sequence encoding the second polypeptide ligated in frame. In some embodiments, the nucleotide sequence encoding the first polypeptide, the nucleotide sequence encoding the self-cleavage peptide, and the nucleotide sequence encoding the second polypeptide are arranged in the direction from 5 ‘to 3’.

As used herein, the “self-cleaving peptide” means a peptide that may achieve self-cleavage within a cell. For example, the self-cleaving peptide may contain a protease recognition site so as to be recognized and specifically cleaved by the protease in the cell.

Alternatively, the self-cleaving peptide may be a 2A polypeptide. The 2A polypeptide is a class of short peptides originated from viruses whose self-cleaving occurs during translation. When two different polypeptides of interest are linked by the 2A polypeptide and expressed in the same reading frame, the two polypeptides of interest are generated at a ratio of nearly 1:1. The commonly used 2A polypeptides may be P2A from porcine techovirus-1, T2A from Thora asigna virus, E2A from equal rhinitis A virus and F2A from foot-and-mouth disease virus. Among them, P2A has the highest efficiency in cleavage, so it is preferable. Various functional variants of these 2A polypeptides are also known in the art, and may also be used in the present invention. In some embodiments, the self-cleavage peptide is P2A as shown in SEQ ID NO:9.

In some embodiments, the gene editing system at least contains an expression construct, which contains a nucleotide sequence encoding the amino acid sequence shown in SEQ ID NO:10 or SEQ ID NO:11.

In another aspect, the present invention provides a gene editing system for editing a target sequence in the genome of a cell, comprising:

i) a polypeptide and/or an expression construct comprising a nucleotide sequence encoding the polypeptide; and

i) a guide RNA and/or an expression construct comprising a nucleotide sequence encoding the guide RNA,

wherein the polypeptide comprises cytosine deaminase, CRISPR nuclease, AP lyase and optionally uracil-DNA glycosylase (UDG), wherein the guide RNA is capable of targeting the polypeptide to the target sequence in the genome of the cell. In some embodiments, the expression construct comprising the nucleotide sequence encoding the polypeptide and the expression construct comprising the nucleotide sequence encoding the guide RNA may be different expression constructs, or may be the same expression construct. In some embodiments, the polypeptide is an isolated polypeptide, and/or the guide RNA is an isolated RNA. In some embodiments, the polypeptide contains the amino acid sequence shown in SEQ ID NO:10 or SEQ ID NO:11.

As used herein, the term “CRISPR nuclease” generally refers to nucleases present in the naturally occurring CRISPR systems, as well as modified forms thereof, variants thereof, or catalytically active fragments thereof. The CRISPR nuclease may recognize, bind, and/or cleave the target nucleic acid structure by interacting with the guide RNA. This term encompasses any CRISPR system based nuclease or functional variant capable of gene editing in the cell. In some embodiments, the functional variant retains its double-stranded cleavage activity, i.e., the ability to form a double-stranded break (DSB) in the target sequence.

The CRISPR nuclease used in the gene editing system of the present invention may be selected from, for example, Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cpf1, C2c1, C2c3 or C2c2 proteins, or functional variants of these nucleases.

In some embodiments, the CRISPR nuclease comprises Cas9 nuclease or a variant thereof. The Cas9 nuclease may be a Cas9 nuclease originated from a different species, such as spCas9 from S. pyogenes. The Cas9 nuclease variant may comprise, for example, a highly specific variant of Cas9 nuclease, such as the Cas9 nuclease variant eSpCas9(1.0) (K810A/K1003A/R1060A), eSpCas9(1.1) (K848A/K1003A/R1060A) of Feng Zhang et al., and the Cas9 nuclease variant SpCas9-HF1 (N497A/R661A/Q695A/Q926A) developed by J. Keith Joung et al. In some specific embodiments, the CRISPR nuclease has the amino acid sequence shown in SEQ ID NO: 1. In some specific embodiments, the CRISPR nuclease comprises an amino acid sequence having at least 80%, at least 85%, at least 90%, at least 95%, at least 96%, at least 97%, at least 98%, at least 99% sequence identity with SEQ ID NO: 1, or having one or more conservative amino acid substitutions relative to SEQ ID NO: 1.

In some embodiments, the CRISPR nuclease may also comprise Cpf1 nuclease or a variant thereof, such as a highly specific variant. The Cpf1 nuclease may be a Cpf1 nuclease originated from different species, such as Cpf1 nuclease originated from Francisella novicida U112, Acidaminococcus sp. BV3L6 and Lachnospiraceae bacterium ND2006.

As used herein, the “cytosine deaminase” refers to a deaminase that may accept single-stranded DNA as a substrate and may catalyze the deamidation of cytidine or deoxycytidine into uracil or deoxyuracil, respectively. Examples of cytosine deaminase include but are not limited to, for example, APOBEC1 deaminase, activation-induced cytidine deaminase (AID), APOBEC3G, CDA1, human APOBEC3A deaminase, APOBEC3B deaminase (e.g., truncated APOBEC3B deaminase). In some embodiments, the cytosine deaminase is human APOBEC3A deaminase, for example, having an amino acid sequence shown in SEQ ID NO: 2. In some specific embodiments, the cytosine deaminase comprises an amino acid sequence having at least 80%, at least 85%, at least 90%, at least 95%, at least 96%, at least 97%, at least 98%, at least 99% sequence identity with SEQ ID NO: 2, or having one or more conservative amino acid substitutions relative to SEQ ID NO: 2. In some embodiments, the cytosine deaminase is truncated APOBEC3B deaminase (APOBEC3Bctd), for example, the amino acid sequence thereof is shown in SEQ ID NO: 7. In some specific embodiments, the cytosine deaminase comprises an amino acid sequence having at least 80%, at least 85%, at least 90%, at least 95%, at least 96%, at least 97%, at least 98%, at least 99% sequence identity with SEQ ID NO: 7%, or having one or more conservative amino acid substitutions relative to SEQ ID NO: 7.

As used herein, uracil-DNA glycocasylase (UDG) or uracil-N-glycosylase (UNG) refers to an enzyme capable of recognizing the U base and remove the N-glycosidic bond of the base to form an apurinic or apyrimidinic site. The UDG may originate from different sources, for example from E. coli. In some specific embodiments, the UDG has the amino acid sequence shown in SEQ ID NO: 3. In some specific embodiments, the DUG comprises an amino acid sequence having at least 80%, at least 85%, at least 90%, at least 95%, at least 96%, at least 97%, at least 98%, at least 99% sequence identity with SEQ ID NO:3, or having one or more conservative amino acid substitutions relative to SEQ ID NO:3.

“AP lyase”, AP endonuclease and “apurinic pyrimidine lyase” are used interchangeably herein, and refer to an enzyme capable of recognizing the apurinic or apyrimidinic site on the nucleic acid and cleaving the nucleic acid. The AP lyase may originate from different sources, for example from E. coli. In some specific embodiments, the AP lyase has the amino acid sequence shown in SEQ ID NO:4. In some specific embodiments, the AP lyase comprises an amino acid sequence having at least 80%, at least 85%, at least 90%, at least 95%, at least 96%, at least 97%, at least 98%, at least 99% sequence identity with SEQ ID NO:4, or having one or more conservative amino acid substitutions relative to SEQ ID NO:4.

As used herein, “gRNA” and “guide RNA” are used interchangeably, and refer to RNA molecule capable of forming a complex with CRISPR nuclease and capable of targeting the complex to the target sequence due to certain complementarity with the target sequence. For example, in a Cas9-based gene editing system, gRNA typically consists of crRNA and tracrRNA molecules that are partially complementary to form a complex, where the crRNA contains a sequence that is sufficiently complementary to the target sequence so as to hybridize to the target sequence and direct the CRISPR complex (Cas9+crRNA+tracrRNA) to specifically bind to this target sequence. However, it is known in the art that single guide RNA(sgRNA) containing both characteristics of crRNA and tracrRNA can be designed. In the Cpf1-based genome editing system, gRNA typically only consists of mature crRNA molecules, where the sequence contained in the crRNA is sufficiently identical to the target sequence so as to hybridize with the complementary sequence of the target sequence and guide the complex (Cpf1+crRNA) to bind specifically with the target sequence. It is within the ability of those skilled in the art to design a suitable gRNA based on the CRISPR nuclease used and the target sequence to be edited.

As used herein, a “target sequence” is a sequence complementary or identical to (depending on different CRISPR nucleases) the guide sequence having about 20 nucleotides contained in the guide RNA. The guide RNA targets the target sequence by base pairing with the target sequence or its complementary strand.

In some embodiments of the invention, the gene editing results in the deletion of one or more nucleotides in the target sequence, preferably results in the deletion of multiple consecutive nucleotides in the target sequence. The type and length of deletion depends on the position of double-stranded break (DSB) caused by CRISPR nuclease and the number and position of cytosine (C) bases present in the target sequence or its complementary sequence. In some embodiments, the length of the deletion does not exceed the length of the target sequence. For example, the deletion may be deletion of about 1-17 nucleotides, such as 10-17 nucleotides, such as 10, 11, 12, 13, 14, 15, 16, 17 nucleotides.

In some embodiments of the invention, the cytosine deaminase is fused to the N terminal of the CRISPR nuclease.

In some embodiments of the invention, the cytosine deaminase, the CRISPR nuclease, the UDG and/or the AP lyase are directly linked.

In some embodiments of the invention, the cytosine deaminase, the CRISPR nuclease, the UDG and/or the AP lyase are linked with linkers. The linkers may be non-functional amino acid sequences of 1-50 (for example, 1, 2, 3, 4, 5, 6, 7, 8, 9, 10, 11, 12, 13, 14, 15, 16, 17, 18, 19, 20 or 20-25, 25-50) or more amino acids, without any secondary or higher structure. For example, the linker may be a flexible linker, such as GGGGS, GS, GAP, (GGGGS)×3, GGS, (GGS)×7, and the like. In some embodiments, the linker contains the amino acid sequence shown in SEQ ID NO:8.

In some embodiments of the invention, the polypeptide of the invention further comprises a nuclear localization sequence (NLS). In general, one or more NLS in the polypeptide should be of sufficient strength to drive the accumulation of the polypeptide in the nucleus of the cell in an amount capable of performing its gene editing function. In general, the strength of nuclear localization activity is determined by the number, position of NLS in the polypeptide, one or more specific NLS used, or a combination of these factors.

In some embodiments of the present invention, the NLS of the polypeptide of the present invention may be at the N terminal and/or C terminal. In some embodiments of the invention, the NLS of the polypeptide of the present invention may be located between the cytosine deaminase, the CRISPR nuclease, the UDG and/or the AP lyase. In some embodiments, the polypeptide comprises about 1, 2, 3, 4, 5, 6, 7, 8, 9, 10, or more NLS. In some embodiments, the polypeptide comprises about 1, 2, 3, 4, 5, 6, 7, 8, 9, 10, or more NLS at or close to the N terminal. In some embodiments, the polypeptide comprises about 1, 2, 3, 4, 5, 6, 7, 8, 9, 10, or more NLS at or close to the C terminal. In some embodiments, the polypeptide comprises a combination of the above, such as comprises one or more NLS at the N terminal and one or more NLS at the C terminal. When there is more than one NLS, each may be selected as not dependent on other NLS.

In general, NLS consists of one or more short sequences of positively charged lysine or arginine exposed on the surface of the protein, but other types of NLS are also known. Non-limiting examples of NLS include: KKRKV (nucleotide sequence 5′-AAGAAGAGAAAGGTC-3′), PKKKRKV (nucleotide sequence 5′-CCCAAGAAGAAGAGGAAGGTG-3′ or CCAAAGAAGAAGAGGAAGGTT), or SGGSPKKKRKV (nucleotide sequence 5′-TCGGGGGGGAGCCCAAAGAAGAAGCGGAAGGTG-3′).

In addition, according to the DNA position to be edited, the polypeptide of the present invention may also include other localization sequences, such as a cytoplasmic localization sequence, a chloroplast localization sequence, a mitochondrial localization sequence, and the like.

In some specific embodiments of the invention, the first polypeptide comprises the amino acid sequence shown in SEQ ID NO:5. In some specific embodiments of the invention, the second polypeptide comprises the amino acid sequence shown in SEQ ID NO:6.

In order to get efficient expression in the cell, in some embodiments of the present invention, the nucleotide sequence encoding the polypeptide is codon optimized for the organism from which the cell to be gene-edited originates.

The codon optimization refers to a method for replacing at least one codon in the natural sequence (for example, about or more than about 1, 2, 3, 4, 5, 10, 15, 20, 25, 50 or more codons) with a codon used more frequently or most frequently in the gene of the host cell, and maintaining the natural amino acid sequence while modifying the nucleic acid sequence to enhance expression in the host cell of interest. Different species exhibit specific preferences for certain codons of specific amino acids. Codon preference (difference in codon usage between organisms) is often related to the translation efficiency of messenger RNA (mRNA), which is considered as depending on the nature of the codon being translated and the availability of the specific transfer RNA (tRNA) molecule. The advantages of the selected tRNA in the cell generally reflect the codons most frequently used for peptide synthesis. Therefore, genes may be tailored to the optimal gene expression in a given organism based on codons optimization. The codon usage tables may be easily obtained, for example, in the codon usage database (“Codon Usage Database”) available at www.kazusa.orjp/codon/, and these tables may be adjusted and applied in different ways. See Nakamura Y. et al., “Codon usage tabulated from the international DNA sequence databases: status for the year 2000”. Nucl. Acids Res., 28: 292 (2000).

The organism from which the cell may be genetically edited by the system of the present invention originates is preferably a eukaryote, including but not limited to, mammals such as human, mouse, rat, monkey, dog, pig, sheep, cattle, cat; poultry such as chicken, duck, geese; and plants, including monocotyledons and dicotyledons, such as rice, corn, wheat, sorghum, barley, soybean, peanut, Arabidopsis, etc.

### III. Method for Modifying the Target Sequence in the Genome of a Cell

In another aspect, the present invention provides a method for modifying a target sequence in the genome of a cell, comprising introducing the gene editing system of the present invention into the cell.

In some embodiments of the invention, the modification results in the deletion of one or more nucleotides in the target sequence, preferably results in the deletion of multiple consecutive nucleotides in the target sequence. In the present invention, the type and length of deletion caused by the deletion depends on the position of double-stranded break (DSB) caused by CRISPR nuclease and the number and position of cytosine (C) bases present in the target sequence or its complementary sequence. In some embodiments, the deletion is within the target sequence. In some embodiments, the modification does not include insertion and/or substitution mutation.

In another aspect, the present invention further provides a method for producing a genetically modified cell, comprising introducing the gene editing system of the present invention into the cell.

In another aspect, the present invention further provides a genetically modified organism, comprising a genetically modified cell or progeny cell thereof produced by the method of the present invention.

In the present invention, the target sequence to be modified may be located at any position in the genome, for example, within a functional gene such as a protein-encoding gene, or for example, may be located in a gene expression regulatory region such as a promoter region or an enhancer region, so as to provide modification of the gene function or modification of gene expression. The modification in the target sequence of the cell may be detected by T7EI, PCR/RE or sequencing methods.

In the method of the present invention, the gene editing system may be introduced into the cell by various methods well known to those skilled in the art.

Methods that may be used to introduce the gene editing system of the present invention into the cell include, but are not limited to: calcium phosphate transfection, protoplast fusion, electroporation, lipofection, microinjection, viral infection (e.g., baculovirus, vaccinia virus, adenovirus, adeno-associated virus, lentivirus and other viruses), gene gun method, PEG-mediated protoplasts transformation, Agrobacterium-mediated transformation.

The cells that may be genetically edited by the method of the present invention may be derived from, for example, mammals such as human, mouse, rat, monkey, dog, pig, sheep, cattle, and cat; poultry such as chicken, duck, geese; and plants, including monocotyledons and dicotyledons, such as rice, corn, wheat, sorghum, barley, soybean, peanut, Arabidopsis, etc.

In some embodiments, the method of the invention is performed in vitro. For example, the cell is an isolated cell, or a cell in an isolated tissue or organ.

In yet other embodiments, the method of the invention may also be performed in vivo. For example, the cell is a cell in an organism, and the system of the present invention may be introduced into the cell in vivo by, for example, a virus or Agrobacterium-mediated method.

### IV. Kit

The present invention further comprises a kit for use in the method of the present invention, the kit comprising the gene editing system of the present invention and instructions for use. The kit generally comprises a label indicating the intended use and/or method of use for the contents of the kit. The term label includes any written or recorded material provided on or with the kit or otherwise provided with the kit.

### EXAMPLES

### Materials and Methods

### 1. Construction of Vector

In order to construct the pA3A-Cas9-UDG and pJIT163-Ubi-AP vectors, the UDG and AP lyase sequences from E. coli were obtained from NCBI (accession numbers AMB53293.1 and WP 115209270.1, respectively), and they were codon optimized for rice and genetic synthesized at GENEWIZ, Inc. (Suzhou). The gene fragment of fusion protein of APOBEC3A, Cas9 and UDG and the gene fragment of AP lyase were finally separately introduced into the pJIT163 vector backbone to obtain pA3A-SpCas9-UDG and pJIT163-Ubi-AP vectors.

In addition, APOBEC3A was fused to the N-terminus of Cas9 with an XTEN linker, UDG was fused to the C-terminus of Cas9, and AP lyase was fused to the C-terminus of UDG using a self-splicing 2A polypeptide (P2A), and the gene fragments of the fusion protein were finally introduced into the pJIT163 vector backbone to construct the transient transformation vector AFID-3. Then the APOBEC3 in AFID-3 was replaced by APOBEC3Bctd (originated from human APOBC3B sequence (accession number is NM 004900.5), which is truncated to obtain the C-terminal functional catalytic domain of APOBEC3B (APOBEC3Bctd)) to construct eAFID-3. In addition, the fusion gene fragments with APOBEC3A were integrated into the pHUE411 skeleton with sgRNA expression components by Gibson method to construct a stable transformation vector pH-AFID-3, which were used for the genetic transformation of rice mediated by Agrobacterium infection.

For key enzyme genes in the synthesis of wheat seed coat pigments (flavanone-3-hydroxylase gene, TaF3H-A1/B1/D1; leucoanthocyanidin reductase gene TaLAR-A1/B1/D1) and its regulatory genes (TaMYB10-A1/B1/D1), plasma membrane kinase associated with disease resistance of a plant (TaPMK-A1/B1/D1), vernalization associated genes (TaVRN1-A1/B1/D1) and gibberellin stimulation regulatory factor genes associated with growth and development (TaGASR6-A1/B1/D1), gene editing target site sequences (sgF3HT4, sgLART4, sgMYBT2, sgPMKT1, sgVRN1T1 and sgGS6T2, see Table 1 for detailed sequence) were obtained respectively, then the sgRNA target site primers were synthesized. Then they were annealed and ligated to the pTaU6-sgRNA vector by using T4 ligase, to obtain pTaU6-sgF3HT4, pTaU6-sgLART4, pTaU6-sgMYBT2, PTaU6-sgPMKT1, pTaU6-sgVRN1T1 and pTaU6-sgGS6T2 vectors respectively.

TABLE 1
sgRNA target primers

| Name of Primer | Forward Primer | Reverse Primer |
|---|---|---|
| sgF3HT4 | CTTGCGCCACGC | AAACAGATCCGG |
| | GGTCCCGGATCT | GACCGCGTGGCG |
| sgLART4 | CTTGCTCGACCG | AAACTAGATCTG |
| | CTTCCAGATCTA | GAAGCGGTCGAG |
| sgMYBT2 | CTTGTGGCTCAA | AAACGGCCGGAG |
| | CTACCTCCGGCC | GTAGTTGAGCCA |
| sgPMKT1 | CTTGCGCGGCGT | AAACCGGCCGGG |
| | CGAACCCGGCCG | TTCGACGCCGCG |
| sgVRN1T1 | CTTGCCTTCTCC | AAACCGAGCGGC |
| | AAGCGCCGCTCG | GCTTGGAGAAGG |
| sgGS6T2 | CTTGTCCTCGTT | AAACGGCACCGC |
| | GCCGGCGGTGCC | CGGCAACGAGGA |

9 endogenous targets were selected from 7 rice genes (OsAAT, OsACC, OsCDC48, OsNRT1.1B, OsPDS, OsGRF1, and OsSPL14/OsIPA1) to construct pOsU3-sgRNA vectors, and 4 endogenous targets were selected from 4 wheat genes (TaF3H, TaGASR6, TaMYB10 and TamiR396) to construct pTaU6-sgRNA vector. See Table 2 for the sequence of all target sites. The sgRNA targeting site primers were synthesized. Then they were annealed and ligated to the sgRNA vectors by using T4 ligase.

TABLE 2
sgRNA target

| sgRNA | Target sequence |
|---|---|
| sgOsAAT | CAAGGATCCCAGCCCCGTGAAGG |
| sgOsACC | TCCACAGCTATCACACCCACTGG |
| sgOsCDC48-T1 | GACCAGCCAGCGTCTGGCGCCGG |
| sgOsCDC48-T2 | CCAGATATCATTGACCCTGCCTT |
| sgOsNRT1.1B | ACTAGATATCTAAACCATTAAGG |
| sgOsPDS | GTTGGTCTTTGCTCCTGCAGAGG |
| sgOsSPL14 | CCAGGCGATCGGATCTCCGGTGG |
| sgOsGRF1-miRT | GAACCGTTCAAGAAAGCCTGTGG |
| sgOsIPA1-miRT | CTCTTCTGTCAACCCAGCCATGG |
| sgTaF3H | CCGAGATCCGGGACCGCGTGGCG |
| sgTaGASR6 | CCCGGCACCGCCGGCAACGAGGA |
| sgTaMYB10 | TGGCTCAACTACCTCCGGCCGGG |
| sgTamiR396 | ACTGTGAACTCGCGGGGATGGGG |

The PAM sequence is shown in bold.

### 2. Isolation and Transformation of Protoplasts (4 Biological Replicates)

2.1 Rice or Wheat Seedling Cultivation

The rice seeds of Zhonghua 11 were rinsed with 75% ethanol for 1 minute, then treated with 4% sodium hypochlorite for 30 minutes, and washed with sterile water more than 5 times. Place on M6 medium for 3-4 weeks, 26° C., protected from light.

The wheat seeds were potted and planted in the cultivation room, and cultured for about 1-2 weeks (about 10 days) at a temperature of 25±2° C., light intensity of 1000 Lx, and light exposure of 14-16 h/d.

2.2 Isolation of Protoplast

(1) Young leaf of rice or wheat was taken, and was cut at the centre part into filaments of 0.5-1 mm with a blade. They were then placed and treated in 0.6 M Mannitol solution for 10 min in the dark, and were then filtered with a filter screen and placed into 50 mL enzymolysis solution (filtered with 0.45 μm filter membrane), which was then evacuated (at a pressure of about 15 Kpa) for 30 min. After removal, they were placed on a shaker (10 rpm) for enzymolysis for 5 h at room temperature. (2) 30-50 mL W5 was added to dilute the enzymolysis product and the enzymolysis solution was filtered with 75 μm nylon filter membrane in a round bottom centrifuge tube (50 mL). (3) At 23° C., 100 g (rcf), it was lifted for 3 times and lowered for 3 times and centrifuged for 3 min, and the supernatant was then discarded. (4) It was then suspended gently with 10 mL W5 and placed on ice for 30 min. The protoplasts were gradually settled and the supernatant was discarded. (5) It was then suspended by adding an appropriate amount of MMG, and then placed on ice until being transformed.

2.3 Protoplast Transformation

(1) 10 μg vectors to be transformed were added respectively to a 2 mL centrifuge tube, and 200 μL of protoplasts was drawn with a sharpened pipette tip after mixing well, which was flicked gently to mix well. It was then added with 250 μL of PEG4000 solution immediately and mixed by flicking gently, and was then induced to conversion at room temperature in the dark for 20-30 min. (2) 800 μL W5 was added (at room temperature) and mixed gently by inverting, and at 100 g (rcf), lifted for 3 time and lowered for 3 times, and centrifuged for 3 min. The supernatant was then discarded. (3) It was then added with 1 mL of W5 and mixed by inverting gently, and was gently transferred to a 6-well plate, which was added with 1 mL of W5 in advance. The 6-well plate was wrapped with tin foil, and incubated at 23° C. in the dark for 48 h.

### 3. Extraction of Protoplast DNA and Amplicon Sequencing Analysis

3.1 Extraction of Protoplast DNA

The protoplast was collected in a 2 mL centrifuge tube, the protoplast DNA (about 30 μL) was extracted by using the CTAB method, with its concentration (30-60 ng/μL) measured by using a NanoDrop ultramicro spectrophotometer, and then stored at −20° C.

3.2 Amplicon Sequencing Analysis

(1) PCR amplification was performed to the protoplast DNA template using genome universal primers. The 20 μL amplification system contains 4 μL 5×Fastpfu buffer, 1.6 dNTPs (2.5 mM), 0.4 μL Forward primer (10 μM), 0.4 μL Reverse primer (10 μM), 0.4 FastPfu polymerase (2.5 U/μL), and 2 μL DNA template (about 60 ng). Amplification conditions: pre-denaturation at 95° C. for 5 min; denaturation at 95° C. for 30 s, annealing at 50-64° C. for 30 s, extension at 72° C. for 30 s for 35 cycles; fully extension at 72° C. for 5 min, and store at 12° C.;

(2) The above amplification product was diluted by 10-fold, and 1 μL was used as the template for the second round of PCR amplification. The amplification primer was a sequencing primer containing Barcode. The 50 μL amplification system contains 10 μL 5×Fastpfu buffer, 4 μL dNTPs (2.5 mM), 1 μL Forward primer (10 μM), 1 μL Reverse primer (10 μM), 1 μL FastPfu polymerase (2.5 U/μL), and 1 μL DNA template. The amplification conditions are as described above, and the number of amplification cycles is 38 cycles.

(3) The PCR products were separated on 2% agarose gel electrophoresis, and AxyPrep™ DNA Gel Extraction kit was used to recover the target fragments. The recovered products were quantitatively analyzed by NanoDrop ultra-micro spectrophotometer. 100 ng of the recovered products were taken respectively and mixed and sent to GENEWIZ, Inc. for amplicon sequencing library construction and amplicon sequencing analysis.

(4) After the sequencing was done, the original data was split according to the sequencing primers, and by using the sgRNA sequence and its flanking sequence as the reference sequences, and the WT as the control, the type and efficiency of gene editing on the different gene targeting sites in the 4 test replicates was comparatively analyzed.

### Example 1. Construction of Gene Editing System (ACD) for Precise Short Fragment Deletion

The single-base editing system has been established in 2016 (Komor et al., 2016; Ma et al., 2016; Nishida et al., 2016). The system uses nCas9 (D10A) to guide the action of cytosine deaminase on the non-complementary strand of a DNA target site, and deaminate the cytosine (C) in a specific region into uracil (U). The uracil (U) will be replaced by thymine (T) in the process of DNA replication, thus achieving accurate single-base replacement of C-to-T. In the repair process of animal and plant organism, the uracil-DNA glycocasylase (UDG) will preferentially recognize the U base and remove the N-glycosidic bond of the base to form an apurinic or apyrimidinic site (AP site), and then repair the U base to the original C base under the action of AP lyase through base excision repair. Therefore, uracil-DNA glycocasylase inhibitor (UGI) is often introduced in a single-base editing system to improve C-to-T editing efficiency.

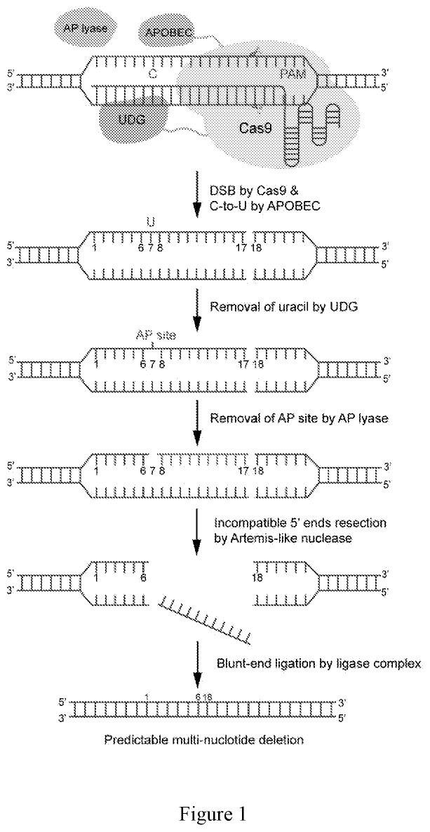

The inventors have surprisingly found that replacing nCas9 of the fusion protein in the single-base editing system with wild-type Cas9 allows the fusion protein to regain the ability to break the DNA double strand, while replacing UGI with UDG to recognize the U base and excise its glycosidic bond to form the AP site, which in turn is recognized by AP lyase, excising the glycosylated U base, which can eventually achieve efficient, accurate and predictable deletion of short fragments in cells. The inventor thus constructed an efficient, accurate and predictable short fragment deletion system (APOBEC3A Coupled Deletion, ACD) consisting of Cas9, APOBEC3A, UDG and AP lyase, where the Cas9 mediates the generation of DSB at the DNA target site, while the APOBEC3A, UDG, and AP lyase mediate multiple gaps at the C base of the non-complementary strand upstream of the DSB, resulting in the deletion of single-stranded DNA fragments on the non-complementary strand, leading to the formation of short double-stranded DNA fragments under the action of DNA repair of an organism (FIG. 1). Without being bound by any theory, the APOBEC3A may efficiently mediate the C-to-U replacement on the non-targeting strand upstream of the DSB, while the UDG and AP lyase mediate the formation of gaps at the U base, resulting in deletion of single stranded DNA fragments on the non-targeting strand. Then, a 5′ overhanging end was formed on the targeting strand. The latter is first recognized and excised by the Artemis-DNA-PK complex during the repair of the non-homologous end of the organism, and further forms short fragment deleted double-stranded DNA at the action of the junctional complex consisting of DNA Ligase IV, XRCC4, XRCC4 analogues (XLF) and their paralogs (PAXX) (Chang et al. 2017).

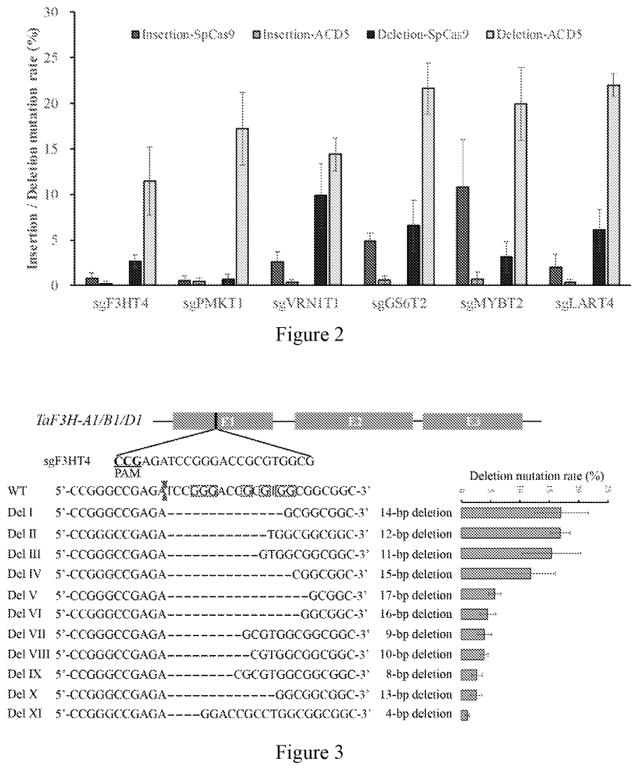

The efficiency of generating insertion and deletion by SpCas9 and ACD were compared and analyzed at the targeted editing sites of sgF3HT4, sgLART4, sgMYBT2, sgPMKT1, sgVRN1T1 and sgGS6T2. The result showed that the Insertion mutation rate generated by the ACD system decreased significantly compared to that by SpCas9, while the Deletion mutation generation rate increased significantly, and the Deletion mutation generation rate was 1.5-23.6 times of that of SpCas9, which fully demonstrated the high efficiency of the ACD system (FIG. 2).

### Example 2. Analysis of the Types of Deletions Generated by the ACD System

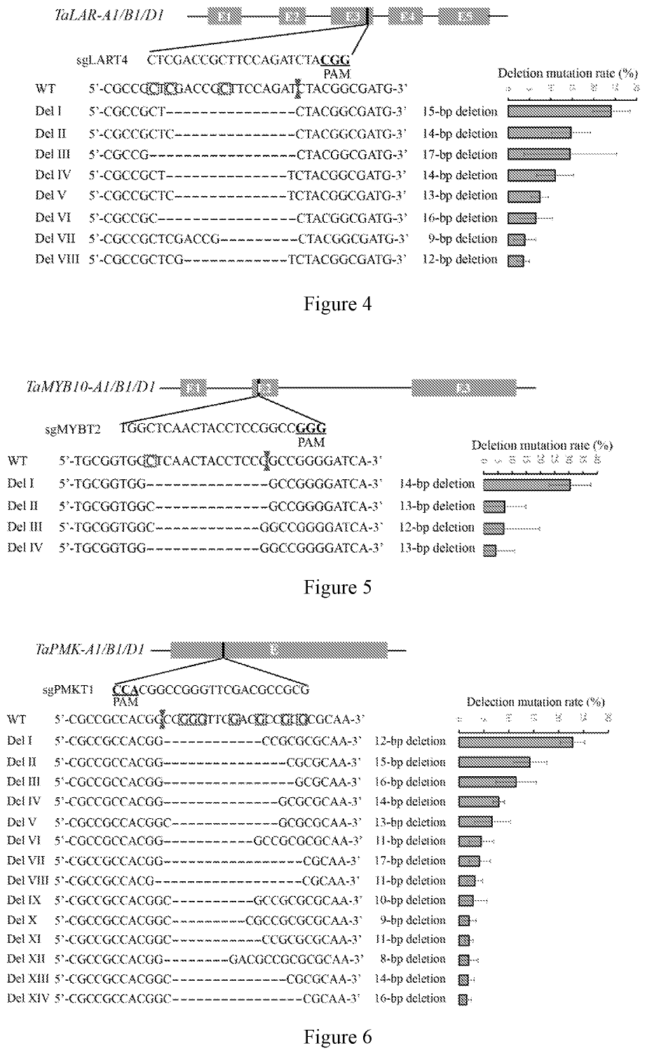

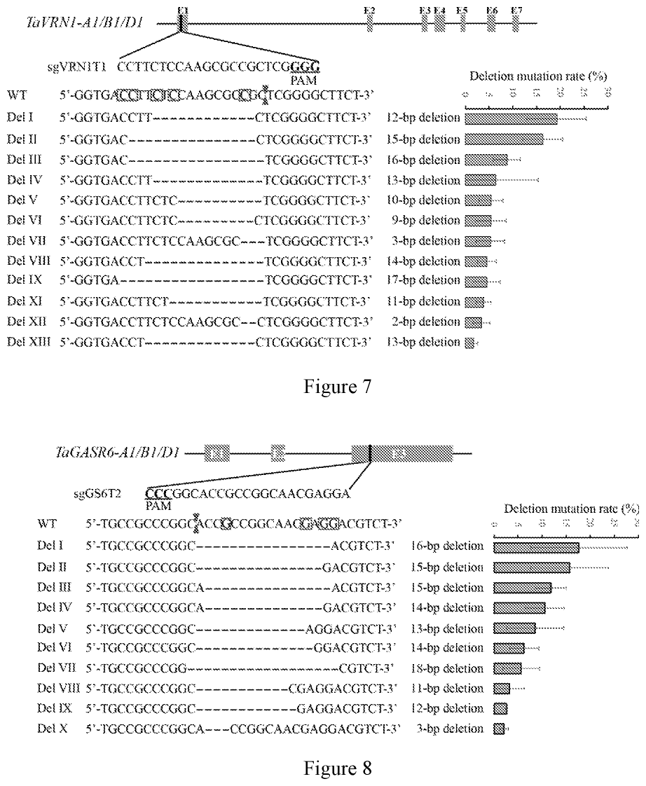

Sequence analysis was carried out for the Deletion mutations generated by the ACD system at different target sites (FIG. 3-8). Except for several types, most mutation types were as expected, and most mutation were Deletion between bases at which APOBEC3A takes effect (NGG (PAM) corresponds to the C base; CCN (PAM) corresponds to the G base) and Cas9 cleavage site. However, as Cas9 has an asymmetry in cutting the double strand, Cas9 will cleave between positions 3-4 or 4-5 near the PAM. In addition, the bases on the non-targeting strand at which APOBEC3A takes effect will use the target strand as a template to form 1-2 bases paired with the complementary strand during the repair process. Therefore, it may also introduce 1-2 bases complementary and paired with the target strand.

The efficiency of ACD system to generate Insertion is very low, but the efficiency of generating Deletion is very high, and Deletion only occurs within the 20-bp protospacer sequence. In these target sites, most of the Deletions have a length of 10-17 nt, and different Deletion types may be stably detected in more than 3 biological replicate experiments, which is impossible by SpCas9 and other tools. It also fully reflects the accuracy and predictability of the ACD system.

### Example 3: Construction of AFID (APOBEC-Cas9 Fusion-Induced Deletion) System

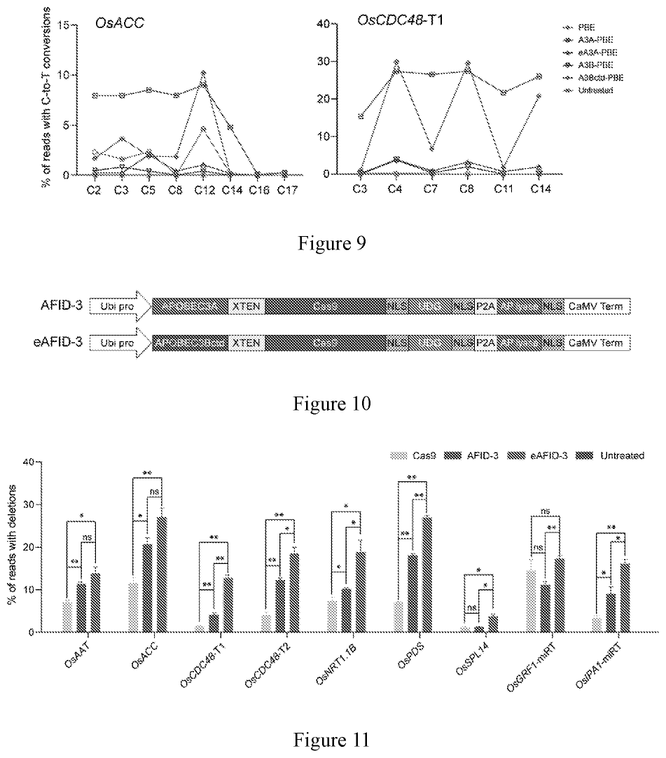

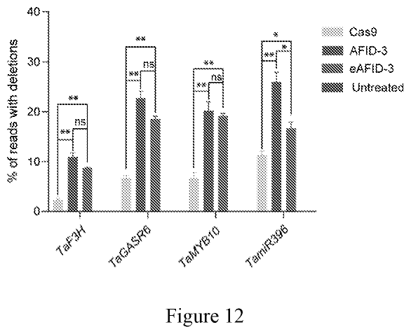

The present invention selects the human APOBEC3A with high deamination activity and wide deamination window to construct the AFID-3 system, and screens a APOBEC3Bctd with higher deamination activity and narrow window to replace the APOBEC3A to construct the eAFID-3 system (FIG. 9 and FIG. 10). Comparative analysis of the deletion efficiencies of Cas9, AFID-3, and eAFID-3 on rice and wheat endogenous gene targets revealed that the efficiency of generating deletion mutations via AFID-3 and eAFID-3 increased significantly compared to Cas9. The average deletion mutation rate was 2.2 times and 2.6 times than that of Cas9, which fully demonstrated the high efficiency of the AFID system.

### Example 4: Analysis of Mutation Types Produced by AFID System

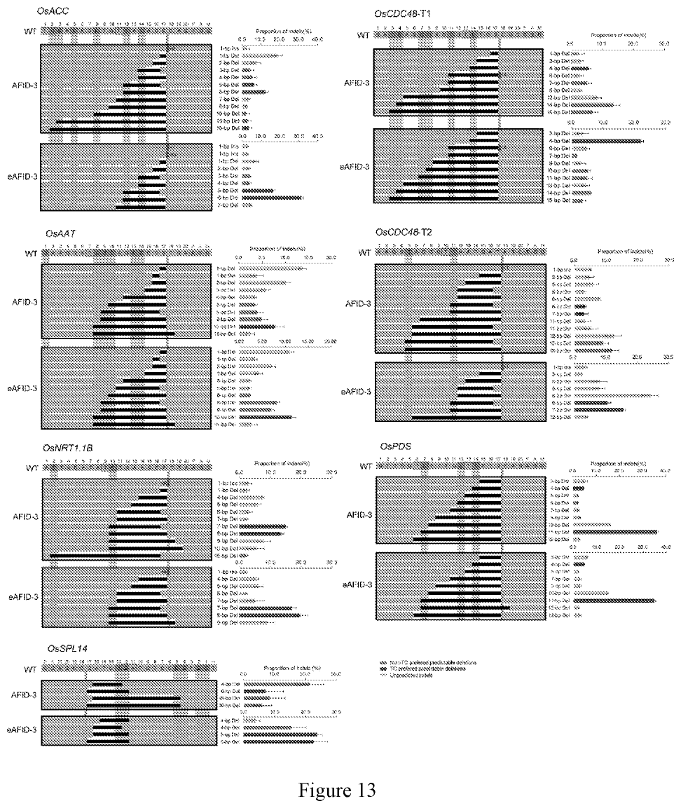

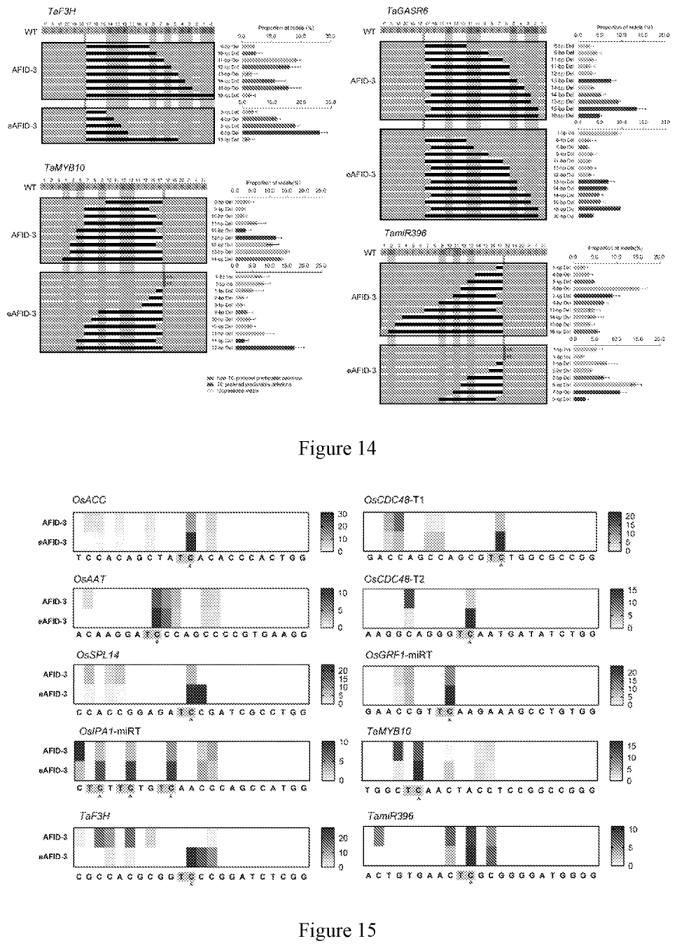

The types and proportions of mutations generated by AFID-3 and eAFID-3 on different endogenous targets were analyzed. The results showed that the length of the deleted fragment mainly depends on the position of the deaminated C nucleotide and its deamination activity. At the target site with strong deamination activity, the mutation type is mainly deletion mutation; but at the target site with weak deamination activity, a certain percentage of insertion mutations will appear. A large proportion of the mutation types are predictable polynucleotide deletion mutations between the C nucleotide where the deaminase works and the Cas9 cleavage site (the cleavage of the double-strand by Cas9 has an asymmetry, resulting in the Cas9 cleavage site appearing between positions 3-4 or between positions 4-5 near the PAM end) (see FIGS. 13 and 14). In addition, it was also found that during the NHEJ repair process, there is a templated insertion of C nucleotides at the deaminated C nucleotides (FIGS. 13 and 14). This is mainly because, in the process of excision of the 5′ protruding terminus of the target strand, DNA polymerase can easily perform base repair on the non-target strand by using the 5′ protruding terminus as templates.

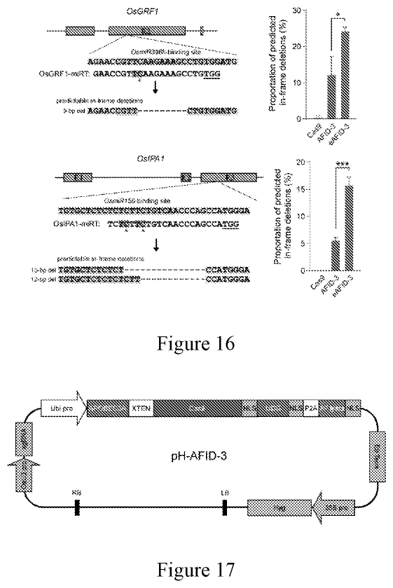

In order to detect the preference of AFID-3 and eAFID-3 for the C base at which the deletion of the predictable fragment starts, the proportion of deletion mutations between AC, TC, CC, and GC motifs and DSBs of different targets were counted. The result showed that AFID-3 can mediate predictable deletion mutations from AC, TC, CC and GC motifs to DSB; eAFID-3 exhibits enhanced TC base preference compared to AFID-3, wherein most of the predictable deletion mutations are deletion mutations from the TC motif to the DSB (FIG. 15). In addition, the types of required predictable deletion mutations in the reading frame and the proportions thereof generating by Cas9, AFID-3 and eAFID-3 at the miR396h binding site of the rice OsGRF1 gene and the miR156 binding site of the OsIPA1 gene were analyzed. The result showed that it is almost difficult for Cas9 to generate the predictable deletion mutation in reading frame; while AFID-3 and eAFID-3 can produce this predictable deletion mutation, but the generation proportion of eAFID-3 is significantly higher than that of AFID-3 (FIG. 16). This also fully reflects the accuracy and predictability of the AFID system.

### Example 5. AFID System Mediates Predictable Polynucleotide Deletion Mutations in Plants

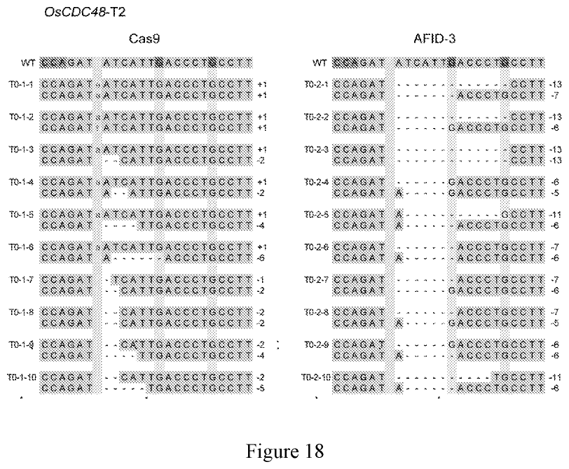

In order to determine whether the AFID system can mediate predictable polynucleotide deletion mutations in plants, two targets (TamiR396 and TaGASR6) were selected on wheat, and Cas9, AFID-3 were delivered into immature wheat embryos with corresponding sgRNA by gene gun bombardment; three targets (OsCDC48-T2, OsSPL14, and OsPDS) were selected on rice to construct the corresponding pH-Cas9 and pH-AFID-3 Agrobacterium vectors (FIG. 17) and rice callus was transformed by Agrobacterium infection. The result showed that among the tested targets, Cas9 did not produce predictable polynucleotide deletion mutants, and the mutation types were mainly 1-bp insertion and 1-3 bp deletion; while AFID-3 produced mostly polynucleotide deletion mutants, the proportion of those predictable accounted for 25.0-55.5% (Table 3, FIG. 18). It can be seen from this that the AFID system can mediate predictable polynucleotide deletion mutations in plants.

TABLE 3
Statistics of predictable deletion mutants in plant generated by AFID-3

| | | | | Number of | Proportion of |
|---|---|---|---|---|---|
| | | | | that contains | predictable |
| | | | Number | predictable | deletion |
| | | Editing | of indel | deletion | mutation |
| Species | Target | tool | mutation | mutation | (%) |
| Wheat | TamiR396 | Cas9 | 0 | 0 | — |
| | | AFID-3 | 8 | 3 | 37.5 |
| | TaGASR6 | Cas9 | 3 | 0 | 0.0 |
| | | AFID-3 | 12 | 3 | 25.0 |
| Rice | OsCDC48-T2 | Cas9 | 44 | 0 | 0.0 |
| | | AFID-3 | 43 | 24 | 55.8 |
| | OsSPL14 | Cas9 | 2 | 0 | 0.0 |
| | | AFID-3 | 27 | 9 | 33.3 |
| | OsPDS | Cas9 | 7 | 0 | 0.0 |
| | | AFID-3 | 6 | 3 | 50.0 |

Sequence List
SpCas9
SEQ ID NO: 1
KDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEV
VKKMKNYWRQLLNAKLITQRKFDNLTKAERGGLSE
LDKAGFIKRQLVETRQITKHVAQILDSRMNTKYDE
NDKLIREVKVITLKSKLVSDFRKDFQFYKVREINN
YHHAHDAYLNAVVGTALIKKYPKLESEFVYGDYKV
YDVRKMIAKSEQEIGKATAKYFFYSNIMNFFKTEI
TLANGEIRKRPLIETNGETGEIVWDKGRDFATVRK
VLSMPQVNIVKKTEVQTGGFSKESILPKRNSDKLI
ARKKDWDPKKYGGFDSPTVAYSVLVVAKVEKGKSK
KLKSVKELLGITIMERSSFEKNPIDFLEAKGYKEV
KKDLIIKLPKYSLFELENGRKRMLASAGELQKGNE
LALPSKYVNFLYLASHYEKLKGSPEDNEQKQLFVE
QHKHYLDEIIEQISEFSKRVILADANLDKVLSAYN
KHRDKPIREQAENIIHLFTLTNLGAPAAFKYFDTT
IDRKRYTSTKEVLDATLIHQSITGLYETRIDLSQL
GGD
APOBEC3A
SEQ ID NO: 2
MEASPASGPRHLMDPHIFTSNFNNGIGRHKTYLCY
EVERLDNGTSVKMDQHRGFLHNQAKNLLCGFYGRH
AELRFLDLVPSLQLDPAQIYRVTWFISWSPCFSWG
CAGEVRAFLQENTHVRLRIFAARIYDYDPLYKEAL
QMLRDAGAQVSIMTYDEFKHCWDTFVDHQGCPFQP
WDGLDEHSQALSGRLRAILQNQGNSGSETPGTSES
ATPES
UDG
SEQ ID NO: 3
ANELTWHDVLAEEKQQPYFLNTLQTVASERQSGVT
IYPPQKDVFNAFRFTELGDVKVVILGQDPYHGPGQ
AHGLAFSVRPGIAIPPSLLNMYKELENTIPGFTRP
NHGYLESWARQGVLLLNTVLTVRAGQAHSHASLGW
ETFTDKVISLINQHREGVVFLLWGSHAQKKGAIID
KQRHHVLKAPHPSPLSAHRGFFGCNHFVLANQWLE
QRGETPIDWMPVLPAESE
AP lyase
SEQ ID NO: 4
MPEGPEIRRAADNLEAAIKGKPLTDVWFAFPQLKP
YQSQLIGQHVTHVETRGKALLTHFSNDLTLYSHNQ
LYGVWRVVDTGEEPQTTRVLRVKLQTADKTILLYS
ASDIEMLTPEQLTTHPFLQRVGPDVLDPNLTPEVV
KERLLSPRFRNRQFAGLLLDQAFLAGLGNYLRVEI
LWQVGLTGNHKAKDLNAAQLDALAHALLEIPRFSY
ATRGQVDENKHHGALFRFKVFHRDGEPCERCGSII
EKTTLSSRPFYWCPGCQH
Exemplary first polypeptide
SEQ ID NO: 5
MEASPASGPRHLMDPHIFTSNFNNGIGRHKTYLCY
EVERLDNGTSVKMDQHRGFLHNQAKNLLCGFYGRH
AELRFLDLVPSLQLDPAQIYRVTWFISWSPCFSWG
CAGEVRAFLQENTHVRLRIFAARIYDYDPLYKEAL
QMLRDAGAQVSIMTYDEFKHCWDTFVDHQGCPFQP
WDGLDEHSQALSGRLRAILQNQGNSGSETPGTSES
ATPESLKDKKYSIGLDIGTNSVGWAVITDEYKVPS
KKFKVLGNTDRHSIKKNLIGALLFDSGETAEATRL
KRTARRRYTRRKNRICYLQEIFSNEMAKVDDSFFH
RLEESFLVEEDKKHERHPIFGNIVDEVAYHEKYPT
IYHLRKKLVDSTDKADLRLIYLALAHMIKFRGHFL
IEGDLNPDNSDVDKLFIQLVQTYNQLFEENPINAS
GVDAKAILSARLSKSRRLENLIAQLPGEKKNGLFG
NLIALSLGLTPNFKSNFDLAEDAKLQLSKDTYDDD
LDNLLAQIGDQYADLFLAAKNLSDAILLSDILRVN
TEITKAPLSASMIKRYDEHHQDLTLLKALVRQQLP
EKYKEIFFDQSKNGYAGYIDGGASQEEFYKFIKPI
LEKMDGTEELLVKLNREDLLRKQRTFDNGSIPHQI
HLGELHAILRRQEDFYPFLKDNREKIEKILTFRIP
YYVGPLARGNSRFAWMTRKSEETITPWNFEEVVDK
GASAQSFIERMTNFDKNLPNEKVLPKHSLLYEYFT
VYNELTKVKYVTEGMRKPAFLSGEQKKAIVDLLFK
TNRKVTVKQLKEDYFKKIECFDSVEISGVEDRFNA
SLGTYHDLLKIIKDKDFLDNEENEDILEDIVLTLT
LFEDREMIEERLKTYAHLFDDKVMKQLKRRRYTGW
GRLSRKLINGIRDKQSGKTILDFLKSDGFANRNFM
QLIHDDSLTFKEDIQKAQVSGQGDSLHEHIANLAG
SPAIKKGILQTVKVVDELVKVMGRHKPENIVIEMA
RENQTTQKGQKNSRERMKRIEEGIKELGSQILKEH
PVENTQLQNEKLYLYYLQNGRDMYVDQELDINRLS
DYDVDHIVPQSFLKDDSIDNKVLTRSDKNRGKSDN
VPSEEVVKKMKNYWRQLLNAKLITQRKFDNLTKAE
RGGLSELDKAGFIKRQLVETRQITKHVAQILDSRM
NTKYDENDKLIREVKVITLKSKLVSDFRKDFQFYK
VREINNYHHAHDAYLNAVVGTALIKKYPKLESEFV
YGDYKVYDVRKMIAKSEQEIGKATAKYFFYSNIMN
FFKTEITLANGEIRKRPLIETNGETGEIVWDKGRD
FATVRKVLSMPQVNIVKKTEVQTGGFSKESILPKR
NSDKLIARKKDWDPKKYGGFDSPTVAYSVLVVAKV
EKGKSKKLKSVKELLGITIMERSSFEKNPIDFLEA
KGYKEVKKDLIIKLPKYSLFELENGRKRMLASAGE
LQKGNELALPSKYVNFLYLASHYEKLKGSPEDNEQ
KQLFVEQHKHYLDEIIEQISEFSKRVILADANLDK
VLSAYNKHRDKPIREQAENIIHLFTLTNLGAPAAF
KYFDTTIDRKRYTSTKEVLDATLIHQSITGLYETR
IDLSQLGGDKRPAATKKAGQAKKKKTRDSGGSANE
LTWHDVLAEEKQQPYFLNTLQTVASERQSGVTIYP
PQKDVFNAFRFTELGDVKVVILGQDPYHGPGQAHG
LAFSVRPGIAIPPSLLNMYKELENTIPGFTRPNHG
YLESWARQGVLUNTVLTVRAGQAHSHASLGWETFT
DKVISLINQHREGVVFLLWGSHAQKKGAIIDKQRH
HVLKAPHPSPLSAHRGFFGCNHFVLANQWLEQRGE
TPIDWMPVLPAESEPKKKRKV
Exemplary second polypeptide
SEQ ID NO: 6
MPEGPEIRRAADNLEAAIKGKPLTDVWFAFPQLKP
YQSQLIGQHVTHVETRGKALLTHFSNDLTLYSHNQ
LYGVWRVVDTGEEPQTTRVLRVKLQTADKTILLYS
ASDIEMLTPEQLTTHPFLQRVGPDVLDPNLTPEVV
KERLLSPRFRNRQFAGLLLDQAFLAGLGNYLRVEI
LWQVGLTGNHKAKDLNAAQLDALAHALLEIPRFSY
ATRGQVDENKHHGALFRFKVFHRDGEPCERCGSII
EKTTLSSRPFYWCPGCQHPKKKRKV
APOBEC3Bctd
SEQ ID NO: 7
MEILRYLMDPDTFTFNFNNDPLVLRRRQTYLCYEV
ERLDNGTWVLMDQHMGFLCNEAKNLLCGFYGRHAE
LRFLDLVPSLQLDPAQIYRVTWFISWSPCFSWGCA
GEVRAFLQENTHVRLRIFAARIYDYDPLYKEALQM
LRDAGAQVSIMTYDEFEYCWDTFVYRQGCPFQPWD
GLEEHSQALSGRLRAILQNQGN
XTEN linker
SEQ ID NO: 8
SGSETPGTSESATPES
P2A
SEQ ID NO: 9
GSGATNFSLLKQAGDVEENPGPPE
AFID-3
SEQ ID NO: 10
MEASPASGPRHLMDPHIFTSNFNNGIGRHKTYLCY
EVERLDNGTSVKMDQHRGFLHNQAKNLLCGFYGRH
AELRFLDLVPSLQLDPAQIYRVTWFISWSPCFSWG
CAGEVRAFLQENTHVRLRIFAARIYDYDPLYKEAL
QMLRDAGAQVSIMTYDEFKHCWDTFVDHQGCPFQP
WDGLDEHSQALSGRLRAILQNQGNSGSETPGTSES
ATPESLKDKKYSIGLDIGTNSVGWAVITDEYKVPS
KKFKVLGNTDRHSIKKNLIGALLFDSGETAEATRL
KRTARRRYTRRKNRICYLQEIFSNEMAKVDDSFFH
RLEESFLVEEDKKHERHPIFGNIVDEVAYHEKYPT
IYHLRKKLVDSTDKADLRLIYLALAHMIKFRGHFL
IEGDLNPDNSDVDKLFIQLVQTYNQLFEENPINAS
GVDAKAILSARLSKSRRLENLIAQLPGEKKNGLFG
NLIALSLGLTPNFKSNFDLAEDAKLQLSKDTYDDD
LDNLLAQIGDQYADLFLAAKNLSDAILLSDILRVN
TEITKAPLSASMIKRYDEHHQDLTLLKALVRQQLP
EKYKEIFFDQSKNGYAGYIDGGASQEEFYKFIKPI
LEKMDGTEELLVKLNREDLLRKQRTFDNGSIPHQI
HLGELHAILRRQEDFYPFLKDNREKIEKILTFRIP
YYVGPLARGNSRFAWMTRKSEETITPWNFEEVVDK
GASAQSFIERMTNFDKNLPNEKVLPKHSLLYEYFT
VYNELTKVKYVTEGMRKPAFLSGEQKKAIVDLLFK
TNRKVTVKQLKEDYFKKIECFDSVEISGVEDRFNA
SLGTYHDLLKIIKDKDFLDNEENEDILEDIVLTLT
LFEDREMIEERLKTYAHLFDDKVMKQLKRRRYTGW
GRLSRKLINGIRDKQSGKTILDFLKSDGFANRNFM
QLIHDDSLTFKEDIQKAQVSGQGDSLHEHIANLAG
SPAIKKGILQTVKVVDELVKVMGRHKPENIVIEMA
RENQTTQKGQKNSRERMKRIEEGIKELGSQILKEH
PVENTQLQNEKLYLYYLQNGRDMYVDQELDINRLS
DYDVDHIVPQSFLKDDSIDNKVLTRSDKNRGKSDN
VPSEEVVKKMKNYWRQLLNAKLITQRKFDNLTKAE
RGGLSELDKAGFIKRQLVETRQITKHVAQILDSRM
NTKYDENDKLIREVKVITLKSKLVSDFRKDFQFYK
VREINNYHHAHDAYLNAVVGTALIKKYPKLESEFV
YGDYKVYDVRKMIAKSEQEIGKATAKYFFYSNIMN
FFKTEITLANGEIRKRPLIETNGETGEIVWDKGRD
FATVRKVLSMPQVNIVKKTEVQTGGFSKESILPKR
NSDKLIARKKDWDPKKYGGFDSPTVAYSVLVVAKV
EKGKSKKLKSVKELLGITIMERSSFEKNPIDFLEA
KGYKEVKKDLIIKLPKYSLFELENGRKRMLASAGE
LQKGNELALPSKYVNFLYLASHYEKLKGSPEDNEQ
KQLFVEQHKHYLDEIIEQISEFSKRVILADANLDK
VLSAYNKHRDKPIREQAENIIHLFTLTNLGAPAAF
KYFDTTIDRKRYTSTKEVLDATLIHQSITGLYETR
IDLSQLGGDKRPAATKKAGQAKKKKTRDSGGSANE
LTWHDVLAEEKQQPYFLNTLQTVASERQSGVTIYP
PQKDVFNAFRFTELGDVKVVILGQDPYHGPGQAHG
LAFSVRPGIAIPPSLLNMYKELENTIPGFTRPNHG
YLESWARQGVLUNTVLTVRAGQAHSHASLGWETFT
DKVISLINQHREGVVFLLWGSHAQKKGAIIDKQRH
EIVLKAPHPSPLSAHRGFFGCNHFVLANQWLEQRG
ETPIDWMPVLPAESEPKKKRKVSAGSGATNFSLLK
QAGDVEENPGPPEGPEIRRAADNLEAAIKGKPLTD
VWFAFPQLKPYQSQLIGQHVTHVETRGKALLTHFS
NDLTLYSHNQLYGVWRVVDTGEEPQTTRVLRVKLQ
TADKTILLYSASDIEMLTPEQLTTHPFLQRVGPDV
LDPNLTPEVVKERLLSPRFRNRQFAGLLLDQAFLA
GLGNYLRVEILWQVGLTGNHKAKDLNAAQLDALAH
ALLEIPRFSYATRGQVDENKHHGALFRFKVFHRDG
EPCERCGSIIEKTTLSSRPFYWCPGCQHPKKKRKV
eAFID-3
SEQ ID NO: 11
MEILRYLMDPDTFTFNFNNDPLVLRRRQTYLCYEV
ERLDNGTWVLMDQHMGFLCNEAKNLLCGFYGRHAE
LRFLDLVPSLQLDPAQIYRVTWFISWSPCFSWGCA
GEVRAFLQENTHVRLRIFAARIYDYDPLYKEALQM
LRDAGAQVSIMTYDEFEYCWDTFVYRQGCPFQPWD
GLEEHSQALSGRLRAILQNQGNSGSETPGTSESAT
PESLKDKKYSIGLDIGTNSVGWAVITDEYKVPSKK
FKVLGNTDRHSIKKNLIGALLFDSGETAEATRLKR
TARRRYTRRKNRICYLQEIFSNEMAKVDDSFFHRL
EESFLVEEDKKHERHPIFGNIVDEVAYHEKYPTIY
HLRKKLVDSTDKADLRLIYLALAHMIKFRGHFLIE
GDLNPDNSDVDKLFIQLVQTYNQLFEENPINASGV
DAKAILSARLSKSRRLENLIAQLPGEKKNGLFGNL
IALSLGLTPNFKSNFDLAEDAKLQLSKDTYDDDLD
NLLAQIGDQYADLFLAAKNLSDAILLSDILRVNTE
ITKAPLSASMIKRYDEHHQDLTLLKALVRQQLPEK
YKEIFFDQSKNGYAGYIDGGASQEEFYKFIKPILE
KMDGTEELLVKLNREDLLRKQRTFDNGSIPHQIHL
GELHAILRRQEDFYPFLKDNREKIEKILTFRIPYY
VGPLARGNSRFAWMTRKSEETITPWNFEEVVDKGA
SAQSFIERMTNFDKNLPNEKVLPKHSLLYEYFTVY
NELTKVKYVTEGMRKPAFLSGEQKKAIVDLLFKTN
RKVTVKQLKEDYFKKIECFDSVEISGVEDRFNASL
GTYHDLLKIIKDKDFLDNEENEDILEDIVLTLTLF
EDREMIEERLKTYAHLFDDKVMKQLKRRRYTGWGR
LSRKLINGIRDKQSGKTILDFLKSDGFANRNFMQL
IHDDSLTFKEDIQKAQVSGQGDSLHEHIANLAGSP
AIKKGILQTVKVVDELVKVMGRHKPENIVIEMARE
NQTTQKGQKNSRERMKRIEEGIKELGSQILKEHPV
ENTQLQNEKLYLYYLQNGRDMYVDQELDINRLSDY
DVDHIVPQSFLKDDSIDNKVLTRSDKNRGKSDNVP
SEEVVKKMKNYWRQLLNAKLITQRKFDNLTKAERG
GLSELDKAGFIKRQLVETRQITKHVAQILDSRMNT
KYDENDKLIREVKVITLKSKLVSDFRKDFQFYKVR
EINNYHHAHDAYLNAVVGTALIKKYPKLESEFVYG
DYKVYDVRKMIAKSEQEIGKATAKYFFYSNIMNFF
KTEITLANGEIRKRPLIETNGETGEIVWDKGRDFA
TVRKVLSMPQVNIVKKTEVQTGGFSKESILPKRNS
DKLIARKKDWDPKKYGGFDSPTVAYSVLVVAKVEK
GKSKKLKSVKELLGITIMERSSFEKNPIDFLEAKG
YKEVKKDLIIKLPKYSLFELENGRKRMLASAGELQ
KGNELALPSKYVNFLYLASHYEKLKGSPEDNEQKQ
LFVEQHKHYLDEIIEQISEFSKRVILADANLDKVL
SAYNKHRDKPIREQAENIIHLFTLTNLGAPAAFKY
FDTTIDRKRYTSTKEVLDATLIHQSITGLYETRID
LSQLGGDKRPAATKKAGQAKKKKTRDSGGSANELT
WHDVLAEEKQQPYFLNTLQTVASERQSGVTIYPPQ
KDVFNAFRFTELGDVKVVILGQDPYHGPGQAHGLA
FSVRPGIAIPPSLLNMYKELENTIPGFTRPNHGYL
ESWARQGVLLLNTVLTVRAGQAHSHASLGWETFTD
KVISLINQHREGVVFLLWGSHAQKKGAIIDKQRHH
VLKAPHPSPLSAHRGFFGCNHFVLANQWLEQRGET
PIDWMPVLPAESEPKKKRKVSAGSGATNFSLLKQA
GDVEENPGPPEGPEIRRAADNLEAAIKGKPLTDVW
FAFPQLKPYQSQLIGQHVTHVETRGKALLTHFSND
LTLYSHNQLYGVWRVVDTGEEPQTTRVLRVKLQTA
DKTILLYSASDIEMLTPEQLTTHPFLQRVGPDVLD
PNLTPEVVKERLLSPRFRNRQFAGLLLDQAFLAGL
GNYLRVEILWQVGLTGNHKAKDLNAAQLDALAHAL
LEIPRFSYATRGQVDENKHHGALFRFKVFHRDGEP
CERCGSIIEKTTLSSRPFYWCPGCQHPKKKRKV

1. A gene editing system for editing a target sequence in the genome of a cell, comprising:
i) a first polypeptide and/or an expression construct comprising a nucleotide sequence encoding the first polypeptide;
ii) a second polypeptide and/or an expression construct comprising a nucleotide sequence encoding the second polypeptide; and
iii) a guide RNA and/or an expression construct comprising a nucleotide sequence encoding the guide RNA,
wherein the first polypeptide comprises a CRISPR nuclease, a cytosine deaminase, and optionally an uracil-DNA glycosylase (UDG), wherein the second polypeptide comprises AP lyase, wherein the guide RNA is capable of targeting the first polypeptide to the target sequence in the genome of the cell.

2. A gene editing system for editing a target sequence in the genome of a cell, comprising:
i) a polypeptide and/or an expression construct comprising a nucleotide sequence encoding the polypeptide; and
ii) a guide RNA and/or an expression construct comprising a nucleotide sequence encoding the guide RNA,
wherein the polypeptide comprises a CRISPR nuclease, a cytosine deaminase, an AP lyase and optionally an uracil-DNA glycosylase (UDG), wherein the guide RNA is capable of targeting the polypeptide to the target sequence in the genome of the cell.

3. The gene editing system of claim 1, wherein the CRISPR nuclease is a Cas9 nuclease, such as spCas9.

4. The gene editing system of claim 1, wherein the cytosine deaminase is APOBEC3A deaminase.

5. The gene editing system of claim 1, wherein the UDG comprises the amino acid sequence shown in SEQ ID NO. 3.

6. The gene editing system of claim 1, wherein the AP lyase comprises the amino acid sequence shown in SEQ ID NO. 4.

7. The gene editing system of claim 1, wherein the first polypeptide comprises the amino acid sequence shown in SEQ ID NO. 5, and the second polypeptide comprises the amino acid sequence shown in SEQ ID NO. 6.

8. A method of producing a genetically modified cell, comprising introducing the gene editing system of claim 1 into the cell.

9. The method of claim 8, wherein the genetic modification is deletion of one or more nucleotides in the target sequence, preferably deletion of multiple consecutive nucleotides.

10. The method of claim 8, wherein the cell is derived from, for example, mammals such as human, mouse, rat, monkey, dog, pig, sheep, cattle, cat; poultry such as chicken, duck, geese; and plants, including monocotyledons and dicotyledons, such as rice, corn, wheat, sorghum, barley, soybean, peanut, Arabidopsis.

11. A kit comprising the gene editing system of claim 1, and instruction for use.


SEQUENCE LISTING (11 sequences)

SEQ ID NO: 1 - protein, 1368 aa, Streptococcus pyogenes
KDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 215 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPES

SEQ ID NO: 3 - protein, 228 aa, Escherichia coli
ANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE

SEQ ID NO: 4 - protein, 263 aa, escherichia coli
MPEGPEIRRAADNLEAAIKGKPLTDVWFAFPQLKPYQSQLIGQHVTHVETRGKALLTHFSNDLTLYSHNQLYGVWRVVDTGEEPQTTRVLRVKLQTADKTILLYSASDIEMLTPEQLTTHPFLQRVGPDVLDPNLTPEVVKERLLSPRFRNRQFAGLLLDQAFLAGLGNYLRVEILWQVGLTGNHKAKDLNAAQLDALAHALLEIPRFSYATRGQVDENKHHGALFRFKVFHRDGEPCERCGSIIEKTTLSSRPFYWCPGCQH

SEQ ID NO: 5 - protein, 1842 aa, Artificial Sequence
  Exemplary first polypeptide
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESLKDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKTRDSGGSANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESEPKKKRKV

SEQ ID NO: 6 - protein, 270 aa, Artificial Sequence
  Exemplary second polypeptide
MPEGPEIRRAADNLEAAIKGKPLTDVWFAFPQLKPYQSQLIGQHVTHVETRGKALLTHFSNDLTLYSHNQLYGVWRVVDTGEEPQTTRVLRVKLQTADKTILLYSASDIEMLTPEQLTTHPFLQRVGPDVLDPNLTPEVVKERLLSPRFRNRQFAGLLLDQAFLAGLGNYLRVEILWQVGLTGNHKAKDLNAAQLDALAHALLEIPRFSYATRGQVDENKHHGALFRFKVFHRDGEPCERCGSIIEKTTLSSRPFYWCPGCQHPKKKRKV

SEQ ID NO: 7 - protein, 197 aa, artificial sequence
  APOBEC3Bctd
MEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 8 - protein, 16 aa, artificial sequence
  XTEN linker
SGSETPGTSESATPES

SEQ ID NO: 9 - protein, 24 aa, artificial sequence
  P2A
GSGATNFSLLKQAGDVEENPGPPE

SEQ ID NO: 10 - protein, 2135 aa, artificial sequence
  AFID-3
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESLKDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKTRDSGGSANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESEPKKKRKVSAGSGATNFSLLKQAGDVEENPGPPEGPEIRRAADNLEAAIKGKPLTDVWFAFPQLKPYQSQLIGQHVTHVETRGKALLTHFSNDLTLYSHNQLYGVWRVVDTGEEPQTTRVLRVKLQTADKTILLYSASDIEMLTPEQLTTHPFLQRVGPDVLDPNLTPEVVKERLLSPRFRNRQFAGLLLDQAFLAGLGNYLRVEILWQVGLTGNHKAKDLNAAQLDALAHALLEIPRFSYATRGQVDENKHHGALFRFKVFHRDGEPCERCGSIIEKTTLSSRPFYWCPGCQHPKKKRKV

SEQ ID NO: 11 - protein, 2133 aa, artificial sequence
  eAFID-3
MEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGNSGSETPGTSESATPESLKDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKTRDSGGSANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESEPKKKRKVSAGSGATNFSLLKQAGDVEENPGPPEGPEIRRAADNLEAAIKGKPLTDVWFAFPQLKPYQSQLIGQHVTHVETRGKALLTHFSNDLTLYSHNQLYGVWRVVDTGEEPQTTRVLRVKLQTADKTILLYSASDIEMLTPEQLTTHPFLQRVGPDVLDPNLTPEVVKERLLSPRFRNRQFAGLLLDQAFLAGLGNYLRVEILWQVGLTGNHKAKDLNAAQLDALAHALLEIPRFSYATRGQVDENKHHGALFRFKVFHRDGEPCERCGSIIEKTTLSSRPFYWCPGCQHPKKKRKV